(19) United States
(12) Patent Application Publication
(10) Pub. No.: US 2013/0101675 A1
(43) Pub. Date: Apr. 25, 2013

(54) Cross-Linked Non-Demineralized Collagenous Biomaterial

(71) Applicants: Nicolaas Duneas, Groenkloof (ZA); Martina Magdel Lutz, Cape Town (ZA)

(72) Inventors: Nicolaas Duneas, Groenkloof (ZA); Martina Magdel Lutz, Cape Town (ZA)

(21) Appl. No.: 13/624,276

(22) Filed: Sep. 21, 2012

Related U.S. Application Data
(63) Continuation-in-part of application No. 11/581,937, filed on Oct. 17, 2006.
Application No. 11/581,937 is a continuation of application No. 10/489,084, filed on Mar. 10, 2005, now abandoned.

(30) Foreign Application Priority Data
Sep. 6, 2001 (ZA) 2001/7385

(51) Int. Cl. A61K 35/32

(52) U.S. Cl. CPC A61K 35/32
USPC 424/549

(57) ABSTRACT

A cross-linked non-demineralized collagenous biomaterial and a method for making and using the same to regenerate tissue are described. The collagenous biomaterial is produced by sourcing a raw material of bone powder and tendon which were not subjected to treatment that would demineralize them, extracting the raw material to form a collagenous biomaterial, and irradiating the collagenous biomaterial with gamma radiation. The collagenous biomaterial is administered to a treatment site by an administration apparatus.

### CROSS-REFERENCE TO RELATED APPLICATION

This application is a continuation-in-part of U.S. patent application Ser. No. 11/581,937 filed Oct. 17, 2006, which is a continuation of U.S. Ser. No. 10/489,084 filed Mar. 10, 2005, which is a U.S. national phase of PCT/1B02/03576 filed Sep. 4, 2002, and claims priority from South African application Serial No, 2001/7385 filed Sep. 6, 2001.

### DISCLOSURE OF THE INVENTION

This invention relates to collagenous biomaterials.

More particularly, the invention relates to a method of producing a cross-linked undemineralized (non-demineralized) collagenous biomaterial, to a cross-linked collagenous undemineralized (non-demineralized) biomaterial produced by the method, to a tissue-regenerating composition, to a tissue-regenerating device, to the use of a substance or composition in the preparation of a medicament for the regeneration of tissue, to a method of regenerating tissue and to a substance or composition for use in a method of regenerating tissue.

The cross-linked collagenous biomaterials of the invention are particularly intended for use in regenerating delivery systems.

The phrase “collagenous biomaterial” as used in this specification means a material which is extracted (normally mechanically and/or by boiling) from non-demineralized bone powder or non-demineralized tendon and which is not subjected to treatment that will demineralize the material, the resulting biomaterial comprises a mixture of Type I collagen together with other components such as native proteins, trace levels of minerals and trace quantities of sugar and carbohydrates.

In the past there have been other collagen-based biomaterial delivery systems (known collagen systems). The state of the art known collagen systems that these are in the form of salt precipitated, purified Type I collagen such as that described in U.S. Pat. Nos. 4,394,370, Jefferies, “Bone Graft Material for Osseous Defects and Method of Making the Same”; 3,955,012, Okamura et al., “Method for Manufacturing Medical Articles Composed of Silicone Rubber Coated with Collagen”; 5,707,962, Chen et al., “Compositions with Enhanced Osteogenic Potential, Method for Making the Same and Therapeutic Uses Thereof,” and 4,789,663, Wallace et al., “Methods of Bone Repair Using Collagen.”

According to one aspect of the invention, there is provided a method of producing a cross-linked collagenous biomaterial, the method including the steps of
(a) producing a collagenous biomaterial by
   (i) sourcing a raw material from the group consisting of mammalian bone powder and tendon and not subjecting the material to treatment which will demineralize the raw material, and
   (ii) extracting the raw material from the group consisting of bone powder and tendon to form a collagenous biomaterial; and
(b) irradiating the thus produced undemineralized collagenous biomaterial with gamma radiation at a dose of between about 20 and 160 kGy to produce the cross-linked collagenous biomaterial.

The collagenous biomaterial may be in the form of a gel. The collagenous biomaterial may be irradiated at a dosage of between about 70 and 90 kGy and preferably at a dosage of about 80 Gy. The method thus includes the step of irradiating the gel to form the cross-linked collagenous biomaterial.

The collagenous biomaterial used in the cross-linking step of the invention may be an extract containing collagen Type I, together with other components such as native proteins, trace levels of minerals and trace quantities of sugars and carbohydrates. It may be produced by extracting undemineralized bone powder. It may, instead, be produced by extracting undemineralized tendon. The bone powder may be defatted, non-demineralized, milled bone powder from human or animal origin.

For example, bone powder may be boiled in water to produce an extract which is rich in Type I collagen. Instead, a mixture of collagen and elastin may be extracted from human tendon by boiling the tendon in an aqueous acidic medium. These extracts contain minerals and other proteins which are co-extracted with the collagen. Typically, the material comprises Type I collagen and other proteins native in bone powder. The collagenous material typically contains Type I collagen in high abundance, lesser quantities of other native proteins, trace levels of minerals (solubilised from bone during the extraction step) and trace quantities of sugars and carbohydrates.

The extraction step may involve extracting the bone powder with hot water or extracting the tendon with hot aqueous acid, e.g. hot aqueous acetic acid, to produce an aqueous extract.

The extraction step may thus be selected from an aqueous extraction step and an aqueous acidic extraction step.

The method may include the step of isolating the collagenous biomaterial, dissolving the collagenous biomaterial in a pre-determined volume of water to produce a solution of the biomaterial and allowing the solution to set to form a gel.

Isolating the non-demineralized collagenous biomaterial may be by evaporating the aqueous extract. The aqueous extract may be concentrated in a microwave heating step.

The gel may be irradiated with a cobalt 60 source, for example by using a Source Nordion C-188 irradiator.

The invention extends to a cross-linked collagenous biomaterial prepared by a method as hereinbefore described. The cross-linked material may be further processed in the wet state by mincing it to form a paste-like material and admixing the paste-like material with substances selected from processed tissue banked bone, insoluble collagenous bone matrix, bone growth inducing factors, cortical or cancellous bone particles, sintered and powdered hydroxyapatites, ceramic powders, demineralized bone particles and mixtures thereof to produce novel delivery systems for tissue regeneration.

The invention thus extends to producing a tissue-regenerating composition which includes a cross-linked collagenous biomaterial by a method as hereinbefore described and at least one component selected from processed bone, insoluble collagenous bone matrix, bone growth inducing factors, cortical bone particles, cancellous bone particles, hydroxyapatites, ceramic powders, demineralized bone particles and mixtures of any two or more thereof.

The bone growth inducing factors may be selected from bone morphogenetic proteins, transforming growth factor beta and combinations thereof.

The collagenous biomaterial may instead be freeze-dried and milled to a known particle size range, and packaged in a syringe either alone or as a composition with one or more of the substances referred to above which may be rehydrated to form an injectable device useful for the treatment of bone defects in a human or animal.

The invention thus extends further to a tissue-regenerating device which includes a tissue-regenerating composition as hereinbefore described and administration means for administering the composition to a treatment site. The administration means may be a syringe.

Instead, the administration means may be in the form of a carrier such as a membrane, sponge or sheet. For example, it may be in the form of a membrane for use in guided tissue regeneration such as periodontal regeneration, or in the form of a haemostatic sponge for use in stopping hemorrhagic trauma of internal organs such as the liver or spleen or in the form of a sheet for use in covering skin burns.

The phrase “collagenous bone matrix” refers to material which is disassociatively extracted with chaotropic agents such as strong urea or guanidinium solutions from milled demineralized bone powder. The phrase “bone morphogenetic proteins” refers broadly to protein morphogens which are known to induce bone formation in primates when delivered to recipient skeletal sites in conjunction with a suitable carrier material. The phrase “Type I collagen” refers to purified preparations of Type I collagen which are more than 98% pure.

According to another aspect of the invention there is provided the use of a substance or composition in the preparation of a medicament for the regeneration of tissue, the substance or composition including a cross-linked collagenous biomaterial prepared by a method as hereinbefore described.

The substance or composition may include at least one component selected from processed bone, insoluble collagenous bone matrix, bone growth inducing factors, cortical bone particles, cancellous bone particles, hydroxyapatites, ceramic powders, demineralized bone particles and mixtures of any two or more thereof.

According to another aspect of the invention there is provided a method of regenerating tissue, the method including the step of administering to a person or animal in need of treatment an effective amount of a substance or composition which includes a cross-linked collagenous biomaterial prepared by a method as hereinbefore described.

The substance or composition may include at least one component selected from processed tissue banked bone, insoluble collagenous hone matrix, bone growth inducing factors, bone differentiating factors, cortical or cancellous bone particles, sintered and powdered hydroxyapatites, demineralized bone particles, ceramic powders and mixtures thereof.

Bone differentiating factors comprise a large family of proteins which function during embryo formation to cause morphogenesis. This is the formation or construction of tissue with form and function and which take on their respective role in the body. Examples are tissues of bone, cartilage, skin, liver, brain and lung. Some of the growth and differentiating factors are redeployed in adults to cause regeneration of tissue via mechanisms closely resembling embryonic differentiation. Examples of these factors are the bone morphogenetic proteins which induce new bone formation when implanted in an adult.

The method may include treating human bone defects by local application of the medicament directly to bone defects, either by implantation or by injection, for example in humans suffering from bone loss resulting from trauma, tumour resection, osteoporosis, tooth extraction, radiation damage or infectious disease such as tuberculosis or periodontitis.

According to another aspect of the invention, there is provided a substance or composition for use in a method of regenerating tissue, the substance or composition comprising a cross-linked collagenous biomaterial prepared by a method as hereinbefore described and the method comprising administering to a person or animal in need of treatment an effective amount of the substance or composition.

The substance or composition may include at least one component selected from processed tissue banked bone, insoluble collagenous bone matrix, bone growth inducing factors, cortical or cancellous bone particles, sintered and powdered hydroxyapatites, demineralized bone particles, ceramic powders and mixtures thereof.

The bone growth inducing factors may be as hereinbefore described.

The cross-linking of collagen has been widely described in the literature as a method useful for improving the physical properties and biocompatibility of collagen. Prior art cross-linking has generally involved reacting collagen with a cross-linking agent such as dimetnyl-3, 3′-dithiobispropionimidate (Charulatha V, Rajaram A J (2001) Dimethyl-3, 3′-dithiobispropionimidate: a novel cross-linking reagent for collagen; Biomedical Materials Research; January; 54 (1): 122-8), or an aldehyde such as gluteraldehyde or formaldehyde. However, a disadvantage of using chemical cross-linking agents is the cytotoxic nature of these compounds. Collagen is a protein and the chemical cross-linking agents used to cross-link collagen have the capacity also to attack other proteins in the body. A requirement for the manufacture of safe collagenous materials for human use is accordingly the removal of residual cross-linking agent from the cross-linked collagen after the cross-linking step.

It is an object of the present invention to provide a novel method for the preparation of cross-linked collagenous biomaterials with useful biological and physical properties.

It has previously been found that collagen molecules are readily damaged by gamma-radiation at dosages commonly used for sterilizing biomedical products. It has been found that irradiating collagen or chemically cross-linked collagen at a dose which is higher than about 10 kGy causes significant damage to both the collagen and the cross-linked collagen. A significant number of peptide bonds are cleaved by the irradiation and this causes considerable changes in the characteristics of the material.

It has been shown (Cheung D T, Perelman N, Tong D, Nimni M E J (1990) The effect of gamma-irradiation on collagen molecules, isolated alpha-chains, and cross-linked native fibers in Biomed Mater Res 1990 May; 24 (5): 581-9.) that the irradiation of purified Type I collagen from collagenous bone matrix results in radiation damage to the extent that the majority of the gel strength and a sticky properties are lost.

### BRIEF DESCRIPTION OF THE DRAWINGS

FIG. 1 is Chart 1 which shows the results of stickiness force determination of Type I collagen compared to collagenous biomaterial, pre- and post high dose gamma irradiation of 120 kGy.

FIG. 2 is Chart 2 which shows the results of rupture force determination of Type I collagen compared to collagenous biomaterial, pre- and post high dose gamma irradiation of 120 kGy.

FIG. 3 is Chart 3 which shows the results of bloom strength of Type I collagen compared to collagenous biomaterial, following high dose gamma irradiation of 120 kGy.

FIG. 4 is Chart 4 which shows the results of viscosity determination of Type I collagen compared to collagenous biomaterial, pre- and post high dose gamma irradiation of 120 kGy.

The invention now provides a novel method for cross-linking collagen without using a cross-linking agent. The invention involves the isolation of a collagenous material which is rich in collagen type I from human tissue banked bone or human bone tendon, refraining from treating the collagenous biomaterial with any step that would demineralize the collagenous biomaterial, the preparation of a gel with a specific range of protein concentration from the isolated collagenous material and the exposure of the gel to gamma irradiation to cause cross-linking. The gel has been found to have a markedly better gel strength and stickiness compared to material prepared from highly purified human Type I collagen (98% pure) using prior art methods.

The collagen biomaterial is a novel material not previously known in published literature. In the published literature the collagenous biomaterial has always been subjected to salt precipitated in order to remove contaminants.

The invention is now described, by way of example, with reference to the accompanying Examples and Tables.

### EXAMPLE 1

Diaphyseal human long bone shafts were cut longitudinally, demarrowed, and cleaned of adhering soft tissues until only the cortical bone remained. In another embodiment of the invention the bone shafts were bovine bone shafts. The cut pieces were reduced in size to small pieces and defatted with 3 to 5 volumes of ethyl ether, dehydrated with 3 volumes of ethyl alcohol and air dried. In another embodiment of the invention the pieces were defatted with a 1:1 mixture of chloroform and methanol. The defatted dried material was milled to a particle size of less than 75 microns. The milled, defatted, dried material was not subjected to any steps which would demineralize the material. The milled, defatted, dried material was boiled in five volumes of purified water in a pressure vessel for one to two hours. In another embodiment of the invention, the dried material was not subjected to any steps which would demineralize the material. The milled, defatted, dried material was boiled in physiological saline solution. In still another embodiment of the invention, the dried material was boiled in an open vessel ensuring replenishment of water lost by evaporation.

The resulting non-demineralized (undemineralized) suspension was filtered using a paper filter and the resulting clarified liquid was concentrated by evaporation on a hot plate to produce the collagenous biomaterial. In another embodiment of the invention, the un-demineralized (non-demineralized) collagenous biomaterial was concentrated by boiling in a microwave oven, dried under vacuum and weighed. An aqueous solution of this material at a concentration of about 15% mass by volume was made up by dissolving the appropriate amount of material in hot water above 80 degrees Celsius until the material had fully dissolved and a clear, pale yellow solution had been produced. In other embodiments of the invention, the concentration of the aqueous solution was between 5% and 20%. The clear solution was decanted into smaller volumes and allowed to set to a solid gel of collagenous biomaterial. In different embodiments of the invention the clear solution was allowed to set at room temperature and in a refrigerator at 4 degrees Celsius.

The undemineralized (non-demineralized) collagenous biomaterial gel was packaged; sealed and irradiated at 80 kGy using a cobalt 60 source from a Sourca Nordion C-188 irradiator to cause cross-linking. In different embodiments, the gel was irradiated at doses which varied between 20 and 160 kGy. The irradiation was carried out at room temperature at ambient conditions of temperature and humidity.

The cross-linked non-demineralized collagenous biomaterial gel was then wet-milled using a blending device which comprises a holding vessel equipped with blades mounted on a rotating drive shaft. Blending was stopped when a fine gelatinous and highly viscous material had been obtained. In different embodiments of the invention, this material was combined with bone chips, DFDBA (defatted, demineralized, freeze-dried bone allograft), collagenous bone matrix or active growth factors to produce compositions. The compositions were packed into syringes and sterilized further by standard doses of gamma irradiation of 2.5 kGy at a temperature of −40 to −20 C. These biological devices were successfully used in sinus lift procedures in humans to regenerate and augment bone in the sinus region for the purposes of obtaining sufficient bone depth in which to place implants intended for tooth prosthesis.

Tables 1A and 1 B set out the results of tests with a composition comprising cross-linked collagenous biomaterial together with cortical or cancellous demineralized or undemineralized bone particles in the promotion of bone formation in human sinus lift procedures.

Maxillary sinus bone changes are shown in mm of new bone generated after 5 and 3 months as judged from before and after radiographs of humans treated with the composition described in Table 1A.

TABLE 1A

| MATERIAL | AMOUNT |
|---|---|
| Milled 16% strength cross-linked 60% | 60% |
| collagenous biomaterial gel | |
| Cortical human bone chips 40% | 40% |

TABLE 1B

| Case number | Trial Period | Amount of | Amount of bone |
|---|---|---|---|
| 1 | 5 | 2 | 9 |
| 2 | 3 | 2 | 8 |

A composition comprising cross-linked collagenous biomaterial together with bone morphogenetic proteins and collagenous bone matrix was found to induce new bone formation when injected into soft tissues of the rodent and bony sites of the human. This composition is described in Table 2.

TABLE 2

| | AMOUNT |
|---|---|
| MATERIAL | milligrams |
| Milled 16% strength cross-linked | 1000 |
| collagenous biomaterial gel | |
| Human demineralized bone matrix | 500 |
| Human bone morphogenetic protein | 0.5-2.5 |

The same material may be injected locally into osteoporotic lesions of the human skeleton to assist in bone regeneration where bone loss has occurred. A composition comprising cross-linked collagenous biomaterial together with apatite powders has been found to be a useful bone filling material.

### EXAMPLE 2

Human achilles tendon was wet milled in 10 volumes of purified water containing 0.2 N acetic acid and autoclaved for 20 minutes in a glass bottle. The supernatant was filtered off and precipitated with 4 volumes of chilled absolute alcohol. The precipitated material was dried in vacuo and reconstituted with purified water at 80 degrees Celsius. In different embodiments of the invention, the amount of water used to reconstitute the precipitated biomaterial was varied to produce a concentration of between 2% and 20%. The material was poured into moulds and allowed to set to a gel at 4 degrees Celsius. The gel which is an undemineralized collagenous biomaterial was then irradiated with a cobalt 60 source at a dosage of at 80 kGy to effect cross-linking. In different embodiments of the invention, the gel was irradiated at dosages which varied between 20 and 160 kGy. The collagenous biomaterial was then wet milled and combined with the biological materials as described in Example 1.

### EXAMPLE 3

### Processing Protocols for Syringed Cross-Linked Collagen Bone

Diaphyseal human long bone shafts were cut longitudinally, demarrowed, and cleaned of soft tissues until only the cortical bone remained. The cut strips were further reduced in size to about 15 mm×15 mm and defatted in three volumes of a 1:1 mixture of chloroform and methanol for 24 hours at room temperature. The solvents were discarded and the bone was defatted in a fresh solution of chloroform and methanol for a further 24 hours. The defatted bone was then dehydrated with 3 volumes of ethyl alcohol for 24 hours and air dried. The defatted, air-dried material was milled to a particle size of less than 125 micron. No further steps were taken to demineralize the collagenous biomaterial.

The fine bone powder was then boiled in Schott high-pressure glass vessels in five volumes of distilled water in a pressure cooker at near maximum temperature for a minimum of 2 hours. After partial cooling, the bottles were vigorously shaken to further separate the mineral and collagenous components, the lids were unscrewed and the mixture was left to settle into two separate components.

The yellow collagenous biomaterial was then drawn off with a syringe and filtered through a paper filter. The filtrate was boiled in a vessel in a microwave oven for several minutes on maximum output, to remove about 75% of the water. A perforated paper filter was used to cover the vessel to prevent loss of biomaterial caused by sputtering during the microwave step. A dark yellow, highly viscous concentrated collagen material was obtained.

This material was gelled at room temperature in 30 cc clear, polyethylene screw-cap containers and then subjected to 80 kGy cross-linking at ambient temperature and humidity. The jars were labeled with the date, dosage and batch number. Irradiation was conducted with a Nordion C-188 Cobalt 60 source.

The cross-linked product was then wet milled with a blending device comprising a holding vessel equipped with blades mounted on a rotating drive shaft made by Braun, deep frozen to −80 degrees Celsius and lyophilized for three days to complete dryness.

The dry, lyophilized product was milled in an IKA milling machine fitted with a 1 mm mesh.

This material was used to prepare injection devices in accordance with the invention.

In each case, the cross-linked undemineralized collagen was dissolved in purified water BP to produce a 20% m/v mixture and rehydrated at 4 Degrees Celsius overnight. The devices are referred to as COB-10, COB-05, COB-02 and COB-01, CAB-02 and CAB-01, and CCAB-0.5, CCAB-02 and CCAB-01.

The ratio of collagenous biomaterial to DFDBA in COB-10, COB-05, COB-02, COB-01, CAB-02 and CAB-01 was 60:40 and the ratio of collagenous biomaterial to DFDBA in CCAB-0,5, CCAB-02 and CCAB-01 was 40: 60. DFDBA refers to demineralized, freeze-dried bone allograft.

The DFDBA particles were sifted very thoroughly to obtain material having a particle size between 250 and 1000 micron. Madison test sieves were used to obtain this particle size by sifting the material three times and discarding any material outside these parameters.

COB-10 The cross-linked undemineralized collagenous biomaterial (60 g) was measured out and wet milled again using the blending device described above. The sifted DFDBA particles were poured into a graded measuring device and compacted lightly by tapping five times on a hard surface.

A compacted volume of 40 cc cortical DFDBA was added to the measured collagenous biomaterial and mixed thoroughly, first with the blending device and then by extrusion from one Promex 50 cc catheter tip syringe to another. A completely smooth and homogeneous mixture was obtained.

This material was then loaded into Becton-Dickinson 10 cc syringes by removing the plunger and extruding 10 cc into the syringe. The plunger was replaced and air removed from the system. The nozzle was then sealed with a tight-fitting cap. The B-D syringes all had nozzle apertures that had been enlarged to 3 mm. The syringes were then packed in two layers of plastic, with instructions for storage and use inserted in the packaging, and deep frozen to −80 degrees Celsius. The syringes and contents were sterilized by Cobalt-60 gamma irradiation at a dose of 25 kGy in the deep frozen state.

COB-05 The process used to prepare COB-10 was repeated but 5 cc of the material was loaded into the Becton-Dickinson 10 cc syringes.

COB-02 The process used to prepare COB-10 was repeated but 2 cc of the material was loaded into Becton-Dickinson 2 cc syringes.

COB-01 The process used to prepare COB-10 was repeated but 1 cc of the material was loaded into Becton-Dickinson 2 cc syringes.

CAB-02 The process used to prepare COB-10 was repeated but 40 cc of cancellous DFDBA was used and 2 cc of the material was loaded into Becton-Dickinson 2 cc syringes.

CAB-01 The process used to prepare COB-10 was repeated but 40 cc of cancellous DFDBA was used and 1 cc of the material was loaded into Becton-Dickinson 2 cc syringes.

CCAB-02 The process used to prepare COB-10 was repeated but 60 cc of cancellous DFDBA was used and 2 cc of the material was loaded into Becton-Dickinson 2 cc syringes.

CCAB-01 The process used to prepare COB-10 was repeated but 40 g of the cross-linked collagenous biomaterial was wet milled, 60 cc of cancellous DFDBA was used and 1 cc of the material was loaded into Becton-Dickinson 2 cc syringes.

CCAB-0, 5 The process used to prepare COB-10 was repeated but 40 g of the cross-linked collagenous biomaterial was wet milled, 60 cc of cancellous DFDBA was used and 0.5 cc of the material was loaded into Becton-Dickinson 2 cc syringes.

CCAB-050 (5 cc) The process used to prepare COB-10 was repeated but 40 g of the cross-linked collagenous biomaterial was wet milled, 60 cc of cancellous DFDBA was used and 5 cc of the material was loaded into Becton-Dickinson 10 cc syringes.

### A Comparison of Gamma Irradiated Known Type I Collagen and Gamma Irradiated Cross Linked Undemineralized Collagen

### Rheology Testing

Furthermore comparative tests were undertaken to establish whether the rheological properties of gamma irradiated (at 120 kGy) collagenous biomaterial improved as compared to gamma irradiated (at 120 kGy) Type I collagenous biomaterial.

Included in the tests undertaken was a stickiness test. The stickiness tests were undertaken by subjecting gamma irradiated (at 120 kGy) collagenous biomaterial and gamma irradiated (at 120 kGy) Type I collagenous biomaterial using a Texture Exponent 32 analytical instrument. It was found that samples of gamma cross-linked collagenous biomaterial gels had a stickiness force of −0.07 g whereas samples of gamma cross-linked Type I collagen gels had a stickiness force of −0.017 g. Thus the gamma irradiation significantly improved cross linking of the undemineralized collagen biomaterial as compared to that of the known Type I collagenous biomaterial which was evident in the results of the stickiness test. The increase in cross-linking due to gamma irradiation increased the stickiness by over four (4) fold in the collagenous biomaterial which was previously defined as not being demineralized as compared to the collagenous biomaterial which had been demineralized.

The gamma irradiated (at 120 kGy) collagenous biomaterial and the gamma irradiated (at 120 kGy) Type I collagen biomaterial were subjected to comparative extrusion function tests. The tests included the steps of blending and dispersing the gamma irradiated Type I collagen and gamma irradiated undemineralized collagen using a blending device comprising a holding vessel equipped with blades mounted on a rotating drive shaft made by Braun for one minute. Thereafter DFDBA was mixed in to the respective blended and dispersed samples and each respective sample was loaded in to a syringe. When the syringe containing the mixture of gamma irradiated Type I collagen and DFDBA was extruded from the syringe the mixture shattered into a non-coherent product containing tiny particles of the shattered gel, with noticeable high degree of syneresis and water loss from the gel. The DFDBA failed to suspend properly, clogging the syringe exit and the formulation was not injectable. Whereas when the syringe containing the mixture of gamma irradiated collagenous biomaterial and DFDBA was extruded from the syringe the mixture formed a highly viscous, coherent and sticky mass when formulated with DBM. This formulation was injectable.

Rupture force comparative tests were undertaken. The rupture force in grams was determined using a 20 mm Perspex probe. The probe was moved a distance of 15 mm at 1 mm per second from the surface of both the gamma irradiated (120 kGy) Type I collagen biomaterial gel and the gamma irradiated (120 kGy) undemineralized collagen biomaterial gel. The rupture force tests showed that the gamma irradiated (120 kGy) Type I collagen biomaterial gel had a rupture force of more than four times that of the gamma irradiated (120 kGy) undemineralized collagen biomaterial gel. This showed that gamma cross-linked Type I collagen Gels are more rigid than the non-demineralized gamma cross-linked Collagenous Biomaterial Gels. Accordingly the malleability is significantly better in the non-demineralized gamma cross-linked collagenous biomaterial gels which allows the gel to be more easily and readily extruded from a syringe in to a void as compared to gamma cross-linked Type I collagen gels which are more rigid.

The inventors have found that when exposing either the (i) collagenous biomaterial as set out in the foregoing description—which has not been purified to a Type I collagen—or (ii) Type I collagen to gamma irradiation, the gamma rays cause two opposing effects on the biomaterials. First the gamma rays cause cross-linking, and second the gamma rays cause scission (the breaking of the molecular bonds within the polypeptides).

The inventors further found that Type I collagen, when gamma irradiated at the doses described, suffers scission damage more predominantly whereas our collagenous biomaterial does not. They found that by using collagenous biomaterial, which essentially is comprised of a crude extract of collagen with the contaminants and minerals found prior to it being demineralized and purified through salt precipitation, has a level of gamma protective factor which purified Type I collagen does not have as one of its properties.

By purifying demineralized collagenous biomaterial to Type I collagen, i.e., by eliminating contaminating components through demineralization, extraction and through salt precipitation of the crude extract and irradiating the Type I collagen with gamma rays, the gamma protective factor is discernibly reduced or lost.

Therefore, when irradiating the Type I collagen the radiation energy damages the molecular bonds in the Type I collagen and gamma cross-linking and scission occurs simultaneously, the scission damage significantly predominates over the cross-linking processes.

By choosing to prepare collagenous biomaterial directly from non-demineralized (or undemineralized bone powder), a putative gamma protective factor is preserved within the crude extract, i.e. the collagenous biomaterial as set out above. The gamma protection ensures that during the gamma irradiation the cross-linking process will dominate over the scission process. Thus, the gel formed after gamma irradiation has improved characteristics, is more manageable and flows homogenously through a syringe as compared to a solution comprising gamma-irradiated Type I collagen. The inventors have found that irradiated Type I collagen suffers from a high degree of water loss (exudation) from the gel, and is therefore prone to gel syneresis (dehydration of the gel). In addition, the gamma-irradiated gel is prone to shear damage during the milling process when made suitable for admixing with demineralized bone matrix (“DBM”) for an injectable composition. The shear damage makes the Type I collagen less useful when this has to be introduced into a bone void by way of a syringe.

By preserving the gamma protection factor as explained earlier, the gamma irradiation leads to increased cross-linking and the reduction of scission.

In contrast, by using purified Type I collagen (free from major contaminants) the gamma-protection is lost. The result of using the purified Type I collagen (the known collagen) as shown in the Jefferies patent supra requires the addition of chemical cross-linking agents such as glutaraldehyde whereas the cross-linking in our product is caused by gamma radiation without the exogenous addition of cross-linking agents. By using the inventive collagenous biomaterial instead of purified Type I collagen, the requirement for exogenously added cross-linking agents is removed in order to form cross-linking in the collagen.

The reduced scission of our collagenous biomaterial as compared to known biomaterial adjuncts (being a Type I collagen) as set out above amounts to a significant improvement in the delivery system for the DBM. The gel performance is improved and allows for the creation of injectable formulations with DBM as it is more manageable and more easily extruded through a syringe without the mixture resisting flow as is the case when using cross-linked Type I collagen. Furthermore, the adjunct as set out when made using cross-linked crude collagenous biomaterial as set out above is not prone to separation and weeping (syneresis) as in the case of cross-linked Type I collagen and, furthermore, there are no chemical agents added in order to create the cross-linking. This requires fewer steps in preparing the presently cross-linked collagenous biomaterial before it may be formulated into extrudable composites with DBM as compared to its purified Type I collagen counterpart.

The cross-linked collagenous biomaterial as described above has been found to be the only biomaterial which is made without the addition of any chemicals or removing the water from the solution in order to be cross-linked, causing our gel to be cost-effective, safer and requiring fewer manipulations.

Furthermore, the physicochemical parameters of the cross-linked collagenous biomaterial gel as described above are significantly different than that of Type I collagen gels. For example, as explained above, the Bloom strength test showed that the elasticity properties of the cross-linked collagenous biomaterial created and composed as described herein is superior to that of known collagenous biomaterial. Various tests were conducted to obtain a comparison between the differences between the cross-linked collagenous biomaterial set forth herein and biomaterial obtained form Type I collagen.

Laboratory experiments were conducted at the Tshwane University of Technology located in Pretoria, South Africa. The laboratory is equipped with state-of-the-art sophisticated equipment and instrumentation to measure rheological, physicochemical and chemical parameters. The cross-linked collagenous biomaterial as described herein was tested comparatively with a biomaterial made from Type I collagen, the collagen as described in the prior art above.

The following tests were taken in addition to the examples set forth above. In order to obtain the collagenous biomaterial and the Type I collagen, the following steps were undertaken:
Step 1: The starting material of human bone (human cortical bone as obtained.
Step 2: The human cortical bone was defatted and dehydrated for 24 hours, first in ten volumes of chloroform: methanol and secondly with ten volumes of absolute alcohol at 8 degrees Celsius.
Step 3: The air-dried human bone was milled in an Alpine hammer mill and sieved to a particle size of below 75 microns.

From this point, the raw bone material that was to be used for both the (i) collagenous biomaterial as described previously herein and (ii) that as would be used for making Type I collagen, which would be used for the comparative tests.

For the preparation of collagenous biomaterial as described previously above, the biomaterial resultant from step 3 above was subjected to the following step:
Step 3a: Milled bone as set out in Step 3 was prepared by cooking up a known mass of the bone powder with 20 volumes of purified water in a pressure cooker, and concentrating the extract to a known solids content by evaporation

For the preparation of substantially pure Type I collagen, the human bone powder was prepared from Step 3 above as follows:
Step 4: Milled bone as set out in Step 3 was demineralized five times with ten volumes of 0.5N hydrochloric acid until a stable acid pH of 1:2 had been achieved, indicating that all of the mineral had been removed.
Step 5: The demineralized bone matrix was neutralized with 0.1N sodium bicarbonate and washed extensively with water.
Step 6: The demineralized bone matrix (DBM) was twice extracted for 16-24 hours with approximately ten volumes of 6M urea, Tris-HCl containing enzyme inhibitors phenylmethylsulfonyl fluoride, benzamidine hydrochloride, n-ethyl maleimide and aminocaproic acid and washed extensively with water. (The resulting collagenous residue is known in the art as insoluble collagenous bone matrix or ICBM.)
Step 6a: The substantially pure Type I collagen was prepared by cooking up a known mass of lyophilized ICBM with 20 volumes of purified water in a pressure cooker and concentrating the collagen to a known solids content.

The purity of the Type I collagen was assessed by denaturing polyacrylamide gel electrophoresis.

Gels of substantially pure Type I collagen (steps 1 to 6a) and collagenous biomaterial (steps 1 to 3a) were prepared with the same solids content for purposes of side-by-side analyses. The solids content for comparative batches were verified using moisture analyses methods.

The collagenous biomaterial (steps 1 to 3a) gel and the Type I collagen (steps 1 to 6a) gel were allowed to form overnight at 4 degrees Celsius and gamma cross-linked under cold but not frozen conditions with 120 kGy irradiation doses.

For ease of reference, the irradiated collagenous biomaterial (steps 1 to 3a) gels shall hereinafter be referred to as “Non-Demineralized Gamma Cross-Linked Collagenous Biomaterial Gels” and the irradiated Type I Collagen (steps 1 to 6a) gels shall hereinafter be referred to as the “Gamma Cross-Linked Type I Collagen Gels.”

### Tests Undertaken

Both native (non-gamma irradiated) and gamma cross-linked gels were tested for rheological (texture) properties and handling/performance.

### (i) Melting Point Tests

The Non-Demineralized Gamma Cross-Linked Collagenous Biomaterial Gels (steps 1-3a plus gamma radiation) and the Gamma Cross-Linked Type I Collagen Gels (steps 1-6a plus gamma radiation) were found to have melting points of greater than 90 degrees Celsius, well above the normal human body temperature of 37.5 degrees Celsius.

Native non-cross-linked gels being the collagenous biomaterial (having been previously defined as being non-demineralized) gels (steps 1-3a) and the Type I collagen gels (steps 1-6a) had melting temperatures below 30 degrees Celsius.

### Conclusion of Melting Point Tests

The advantage of the change in melting point is significant. When the cross-linked biomaterial, either Non-Demineralized Gamma Cross-Linked Collagen Biomaterial Gels (steps 1-3a plus gamma radiation) or the Gamma Cross-Linked Type I Collagen Gels (steps 1-6a plus gamma radiation) are used as a vehicle for allograft products as DBM, the implant vehicle must not melt at 37.5 degrees Celsius, otherwise it would be prone to flow or migrate away from the site of implantation in the human body. The human body has a physiological homeostatic temperature of 37.5 degrees Celsius.

It was found that non-irradiated gels would melt above 20 degrees Celsius. Irradiated gels had not melted at temperatures as high as 90 degrees Celsius, which made the respective gamma irradiated samples acceptable for vehicles for allograft products whereas the non-gamma-irradiated gels were not acceptable for such use.

### (ii) Stickiness Tests

Non-Demineralized Gamma Cross-Linked Collagenous Biomaterial Gels had together with its non-cross-linked counterparts (steps 1-3a) (i.e. non-gamma irradiated) and Gamma Cross-Linked Type I Collagen Gels and its non-irradiated counterpart (steps 1 to 6a) (i.e. non-gamma irradiated) were tested for stickiness.

The stickiness tests were taken by using a Texture Exponent 32 analytical instrument.

### Results of Stickiness Tests

Non-Demineralized Gamma Cross-Linked Collagenous Biomaterial Gels had a stickiness force of −0.07 (see Col-Biom 120 kGy below);

Collagenous biomaterial (previously defined as being non-demineralized) gels (steps 1 to 3a) had a stickiness force of −0.01 (see Col-Biom 0 kGy);

Gamma Cross-Linked Type I Collagen Gels had a stickiness force of −0.017 (see Type I collagen 120 kGy below); and

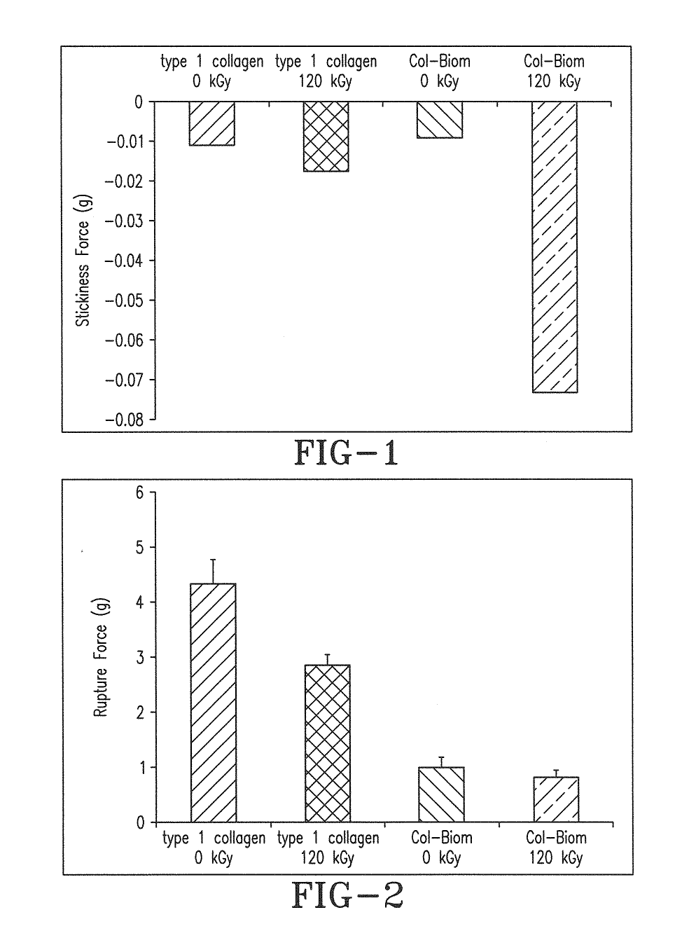

Type I collagen gels (steps 1 to 6a) (i.e. non-gamma irradiated) had a stickiness force of −0.011 (see Type I collagen 0 kGy FIG. 1).

### Conclusion of Stickiness Tests

Gamma irradiation had a striking effect on the stickiness of the collagenous biomaterial gels (steps 1 to 3a) (Chart 1 of FIG. 1) increasing stickiness force measured in grams by 7.72 times. In marked contrast, the stickiness of substantially Type I collagen gels was increased only by 37% as opposed to 680% increase measured for the collagenous biomaterial.

(iii) Extrusion Function Test

The Non-Demineralized Gamma Cross-Linked Collagenous Biomaterial Gels and the Gamma Cross-Linked Type I Collagen Gels were blended and dispersed using a household-type 4185545 Braun blender for one minute.

Aliquots of 50 ml of Non-Demineralized Gamma Cross-Linked Collagenous Biomaterial Gels were mixed with defatted, demineralized, freeze-dried bone allograft (DFDBA), an allograft material commonly used as a bone void filler, to form a mass; and Gamma Cross-Linked Type I Collagen Gels were mixed with demineralized bone matrix DFDBA taken from the same source, to form a mass.

The biomaterial mass was in each case loaded into a syringe and the extrusion or ejection performance noted.

### Results of Extrusion Function Test

The gamma cross-linked Type I collagen gels shattered immediately into a non-coherent product containing tiny particles of the shattered gel, with noticeable high degree of syneresis and water loss from the gel. The DFDBA failed to suspend properly, clogging the syringe exit. This formulation was not injectable.

The Non-Demineralized Gamma Cross-Linked Collagenous Biomaterial Gels—as described herein—formed a highly viscous, coherent and sticky mass when formulated with DFDBA. This formulation was injectable.

### Conclusion of Extrusion Function Test

The Non-Demineralized Gamma Cross-Linked Collagenous Biomaterial Gel could be mixed with bone matrix (DBM) to form an extrudable, injectable mass. In contrast, the Gamma Cross-Linked Type I Collagen Gels failed to perform in combination with DBM to form an injectable biomaterial, and the creation of injectable allogenic DMB product for medical use was only feasible using the Non-Demineralized Gamma Cross-Linked Collagenous Biomaterial Gels as described in our above patent.

### (iv) Rupture Force Tests and Bloom Strength Measurements

### Rupture Force Tests

Non-Demineralized Gamma Cross-Linked Collagenous Biomaterial Gels together with its non-cross-linked counterparts (steps 1 to 3a) (i.e. non-gamma irradiated) and Gamma Cross-Linked Type I Collagen Gels and its non-irradiated counterpart (steps 1 to 6a) (i.e. non-gamma irradiated) were subjected to rupture force test.

Rupture force in grams was determined using a 20 mm Perspex probe. The probe was moved a distance of 15 mm into the gel from the surface of the respective gels at 1 mm per second.

### Results of Rupture Force Tests

The rupture force measured for Type I collagen gels (steps 1 to 6a) (i.e. non-gamma irradiated) was higher than that for collagenous biomaterial gels (steps 1 to 3a) (i.e. non-gamma irradiated), and this trend continued after gamma-irradiation at 120 kGy (Chart 2 of FIG. 2).

The rupture force for Gamma Cross-Linked Type I Collagen Gels was substantially higher than the Non-Demineralized Gamma Cross-Linked Collagenous Biomaterial Gels (Chart 2 of FIG. 2).

This in turn proved that Gamma Cross-Linked Type I Collagen Gels and Type I collagen gels (steps 1 to 6a) (i.e. non-gamma irradiated) are a tougher, more tightly cross-linked substance and are more rigid and less viscous than Non-Demineralized Gamma Cross-Linked Collagenous Biomaterial Gels together with collagenous biomaterial gels (steps 1 to 3a) (i.e. non-gamma irradiated).

### Bloom Strength Measurements

The Bloom strength measurements were undertaken to corroborate the Rupture Force Test experiment. Bloom strength is a measure of the toughness of the respective gels before rupture.

The Bloom strength was also higher in the Gamma Cross-Linked Type I Collagen Gels as compared to Non-Demineralized Gamma Cross-Linked Collagenous Biomaterial Gels (see Chart 3 of FIG. 3).

There was an almost four-fold reduction in Bloom strength from 3.9 g down to 1.02 g (a loss of 74% suffered by the irradiation of the Type I collagen gels.

The Bloom strength reduction of Non-Demineralized Gamma Cross-Linked Collagenous Biomaterial Gels was a 36% loss in grams as compared to collagenous biomaterial gels (steps 1-3a) (i.e. non-gamma irradiated).

The Bloom strength of Non-Demineralized Gamma Cross-Linked Collagenous Biomaterial Gels was about one-third of the Bloom strength of the Gamma Cross-Linked Type I Collagen Gels.

### Conclusion of the Rupture Test and the Bloom Strength Measurement

In all cases, gamma irradiation decreased rupture force (Chart 2 of FIG. 2) and Bloom strength (Chart 3 of FIG. 3) when compared to native non-irradiated gels.

The Bloom strength was significantly higher in the Gamma Cross-Linked Type I Collagen Gels than in the Non-Demineralized Gamma Cross-Linked Collagenous Biomaterial Gels—as described above.

The results show that the Gamma Cross-Linked Type I Collagen Gels are more rigid than the Non-Demineralized Gamma Cross-Linked Collagenous Biomaterial Gels. Therefore, it may be interpreted that the malleability is significantly better in the inventive Gamma Cross-Linked Type I Collagen Gels which allows the gel to be more easily and readily extruded from a syringe into a void as compared to Gamma Cross-Linked Type I Collagen Gels which are more rigid.

### (v) Viscosity Tests

Non-Demineralized Gamma Cross-Linked Collagenous Biomaterial Gels together with its non-cross-linked counterparts (steps 1 to 3a) (i.e. non-gamma irradiated) and Gamma Cross-Linked Type I Collagen Gels and its non-irradiated counterpart (steps 1 to 6a) (i.e. non-gamma irradiated) were tested for viscosity.

### Results of Viscosity Tests

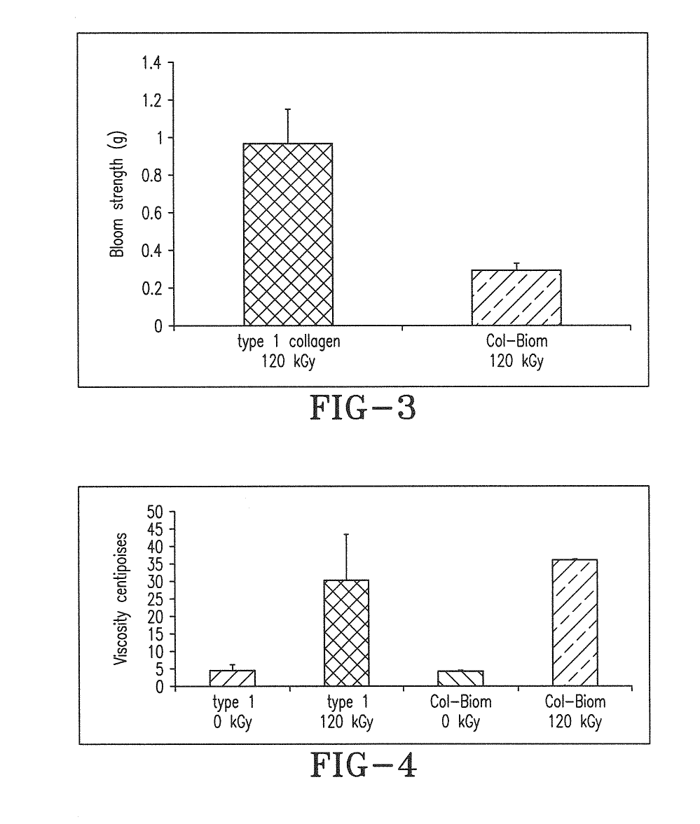

Gamma irradiation increased the viscosity of both the milled biomaterials (see Chart 4 of FIG. 4). The viscosity of the Non-Demineralized Gamma Cross-Linked Collagenous Biomaterial Gels was higher than that of the Gamma Cross-Linked Type I Collagen Gels.

Furthermore, the cross-linking of the Non-Demineralized Gamma Cross-Linked Collagenous Biomaterial Gels was much more consistent judging from the much smaller standard deviation of the data (Chart 4 of FIG. 4) than that of Gamma Cross-Linked Type I Collagen Gels. The standard deviation (statistical variability) of the Gamma Cross-Linked Type I Collagen Gels data set was 28 times higher than that of the gamma cross-linked collagenous biomaterial gels data set.

### Conclusion of Viscosity Test

There was a high degree of unpredictability and variance (shown as an error bar above results for the Type I 120 kGy bar as shown in the Chart 4 of FIG. 4 graph) in the viscosity of Gamma Cross-Linked Type I Collagen Gels when compared to the viscosity of Non-Demineralized Gamma Cross-Linked Collagenous Biomaterial Gels, as set out previously herein.

### Summary of the Rheological Tests Combined

From the tests conducted we have clearly shown that there are significant advantages in the rheology of the Non-Demineralized Gamma Cross-Linked Collagenous Biomaterial Gels as compared to (i) collagenous biomaterial gels (steps 1 to 3a) (i.e. non-gamma irradiated) and (ii) Gamma Cross-Linked Type I Collagen Gels and (iii) Type I collagen gels (steps 1 to 6a) (i.e. non-gamma irradiated).

The tests showed that the gamma cross-linked collagenous biomaterial gels meet the requirements for the melting point suitability, in other words they will not melt when used in a void in the human body and thereby flow away and not remain in the desired location.

The tests established that the Non-Demineralized Gamma Cross-Linked Collagenous Biomaterial Gels obtained the best results on the stickiness tests showing that the gels are most advantageous for their required use, being injectable by means of a syringe.

The Non-Demineralized Gamma Cross-Linked Collagenous Biomaterial Gels, when milled into a processed biomaterial, did not shear as significantly in the product performance tests as compared to Gamma Cross-Linked Type I Collagen Gels. Therefore, the Non-Demineralized Gamma Cross-Linked Collagenous Biomaterial Gels, when used as a suspension agent carrying DBM, are better suited to extrusion through a syringe than known cross-linked Type I collagen gels.

The Non-Demineralized Gamma Cross-Linked Collagenous Biomaterial Gels are softer and less rigid than the Gamma Cross-Linked Type I Collagen Gels, and this again shows an improved delivery of the Non-Demineralized Gamma Cross-Linked Collagenous Biomaterial Gels as compared to Gamma-Cross-Linked Type I Collagen Gels.

The Non-Demineralized Gamma Cross-Linked Collagenous Biomaterial Gels, when used as a suspension agent carrying DBM, were also found to be the least viscous, and again this would improve the delivery of Non-Demineralized Gamma Cross-Linked Collagenous Biomaterial Gels, when used as a suspension agent carrying DBM as compared to the other gels tested.

### IN CONCLUSION

The use of the Non-Demineralized Gamma Cross-Linked Collagenous Biomaterial Gels, when used as a suspension agent, showed an unobvious, beneficial difference as compared to that made from gamma-irradiated Type I collagen (i.e. purified Type I collagen from a demineralized bone matrix). Collagenous biomaterial prepared directly from non-demineralized bone powder (the present gamma cross-linked collagenous biomaterial) results in a material that is substantially different to the material of the current art. The Non-Demineralized Gamma Cross-Linked Collagenous Biomaterial Gel lends itself to the creation of novel composite allograft biomaterials that can perform more advantageously than those known in the art. The advantages include that they can be extruded and injected for precise delivery, they resist irrigation due to the increased stickiness and cohesiveness at the implantation site, and they do not melt at body temperature (37.5 degrees Celsius) and do not flow away, thereby improving the handling characteristics in clinical contexts.

Furthermore, the reason for these improvements is believed to be attributed to the gamma radiation protective factor preserved in the non-demineralized collagenous biomaterial. Whereas when the pure Type I collagen is manufactured from demineralized source material and purified by salt precipitation, the gamma radiation protection factor is discernibly lost. This lowered gamma protection radiation factor reduces the cross-linking which naturally occurs through gamma irradiation.

In the past Gamma irradiation of a collagenous biomaterial was commonly used for its sterilizing properties. The inventors have found that preserving and including the natural contaminants found by direct extraction of collagenous biomaterial from the non-demineralized bone powder enables the successful cross-linking of the collagenous biomaterial with higher than normal doses of irradiation.

The method disclosed herein has proven to create a more effective delivery system for DBM than previous methods, which generally included chemical cross-linking.

It is an advantage of the invention illustrated that the cross-linking method allows biomaterial to be cross-linked by gamma irradiation without any significant damage to the collagen molecule. Prior art methods used for the cross-linking of collagen by gamma irradiation have resulted in extensive damage to the collagen molecule and an accompanying change in the properties of the molecule. The invention therefore provides a method for the cross-linking of undemineralized collagenous biomaterial without the use of chemical cross-linking agents and the accompanying cytotoxic dangers associated with chemically cross-linked collagen. It is not clear why the collagen in the gel is not damaged by gamma irradiation and the applicant believes that other components present in the undemineralized collagenous biomaterial extract may play a role in reducing or preventing irradiation damage.

The invention has been described in detail, with particular emphasis on the preferred embodiment thereof, but variations and modifications within the spirit and scope of the invention may occur to those skilled in the art to which the invention pertains.

The embodiments of the invention in which an exclusive property or privilege is claimed are defined as follows:

1. An irradiated cross-linked collagenous biomaterial produced using a method including the following steps:
(a) producing a collagenous biomaterial by
(i) sourcing a raw material from the group consisting of mammalian bone powder and tendon and not subjecting the material to treatment which will demineralize the raw material, and
(ii) extracting the raw material from the group consisting of bone powder and tendon to form a collagenous biomaterial, and
(b) irradiating the thus produced collagenous biomaterial with gamma radiation at a dose of between about 20 and 160 kGy.

2. A collagenous biomaterial produced by the method as claimed in claim 1, in which the collagenous biomaterial is irradiated at a dosage of between 70 and 90 kGy.

3. A collagenous biomaterial produced by the method as claimed in claim 2, in which the collagenous biomaterial is irradiated at a dosage of about 80 kGy.

4. A collagenous biomaterial produced by the method as claimed in claim 1, in which the collagenous biomaterial is irradiated with a cobalt 60 source.

5. The composition of claim 1, wherein the steps of producing a collagenous biomaterial comprises (i) sourcing a raw material from mammalian bone powder, and (ii) extracting the raw material from bone powder.

6. A composition comprising:
a cross-linked collagenous biomaterial having been irradiated with gamma radiation at a dose of between about 20 and 160 kGy,
and at least one component selected from the group consisting of processed bone, insoluble collagenous bone matrix, bone growth inducing factors, cortical bone particles, cancellous bone particles, hydroxyapatites, ceramic powders, demineralized bone particles and mixtures of any two or more thereof.

7. A composition as claimed in claim 6, in which the bone growth inducing factors are selected from bone morphogenetic proteins, transforming growth factor beta and combinations thereof.

8. Treatment and administration apparatus for inducing tissue regeneration, said treatment and administration apparatus comprising:
a cross-linked collagenous biomaterial having been irradiated with gamma radiation and at least one component selected from the group consisting of processed bone, insoluble collagenous bone matrix, bone growth-inducing factors, cortical bone particles, cancellous bone particles, hydroxyl-apatites, ceramic powders, demineralized bone particles and mixtures of at least two of said components; and
administration apparatus for administering said gamma irradiated cross-linked collagenous biomaterial and said at least one component to a treatment site.

9. Treatment and administration apparatus as claimed in claim 8, wherein the administration apparatus is a syringe.

10. Treatment and administration apparatus as claimed in claim 8, wherein the administration apparatus is a membrane for implantation at the treatment site.

11. Treatment and administration apparatus as claimed in claim 8, wherein the administration apparatus is a haemostatic sponge.

12. Treatment and administration apparatus as claimed in claim 8, in which the administration apparatus is a skin-covering sheet.

13. Treatment and administration apparatus according to claim 8 wherein said gamma irradiated cross-linked collagenous biomaterial is gamma irradiated with a gamma irradiation dose of between about 20 and 160 kGy.

14. Treatment and administration apparatus according to claim 8 wherein said gamma irradiated cross-linked collagenous biomaterial is gamma irradiated with a gamma irradiation dose of between 70 and 90 kGy.

15. A cross-linked collagenous biomaterial produced by the method of:
(a) using a starting material wherein the starting material is not demineralized, produced by a method including the following steps of:
(i) sourcing a raw starting material from the group consisting of mammalian bone powder and tendon, and not subjecting the raw starting material to treatment which will demineralize the raw starting material, and
(iii) extracting the raw starting material from the group consisting of bone powder and tendon to form a collagenous biomaterial, and
(b) irradiating the thus produced collagenous biomaterial with gamma radiation at a dose of between about 20 and 160 kGy to produce the cross-linked collagenous biomaterial.

16. A method of producing an irradiated cross-linked collagenous biomaterial, the method including the following steps:
(a) producing a collagenous biomaterial by
(i) sourcing a raw material from a group comprising undemineralized bone powder and undemineralized tendon, and not subjecting raw material to treatment which will demineralize the raw material, and
(ii) extracting the raw material from the group comprising undemineralized bone powder and undemineralized tendon to form a collagenous biomaterial, and
(b) irradiating the collagenous biomaterial with gamma radiation at a dose of between about 20 and 160 kGy to produce the irradiated cross-linked collagenous biomaterial.

17. A method of producing an irradiated cross-linked undemineralized collagenous material according to claim 16 wherein the bone powder is mammalian bone powder.

18. A method as claimed in claim 16, wherein the step of producing a collagenous biomaterial comprises isolating the collagenous biomaterial, dissolving collagenous material in water to produce a solution of the collagenous material, and allowing the solution to set to form a gel.

19. A method as claimed in claim 16, wherein the step of irradiating the collagenous biomaterial comprises irradiating the collagenous biomaterial with gamma radiation at a dosage of between 70 and 90 kGy.

20. A method as claimed in claim 19, in which the step of irradiating the collagenous biomaterial comprises irradiating the collagenous biomaterial at a dosage of about 80 kGy.

21. A method as claimed in claim 16, wherein the step of extracting a raw material comprises extracting bone powder to form the collagenous biomaterial.

22. A method as claimed in claim 16, wherein the step of extracting a raw material comprises extracting tendon to form a collagenous biomaterial.

23. A method as claimed in claim 21, wherein the bone powder is defatted, dehydrated, milled human bone powder.

24. A method as claimed in claim 21, in which the extraction is selected from an aqueous extraction and an aqueous acidic extraction.

25. A method as claimed in claim 16, wherein the step of irradiating the collagenous biomaterial comprises irradiation with a cobalt 60 source.

26. A cross-linked collagenous biomaterial produced by a method as claimed in claim 16.

27. A method of regenerating tissue, the method including the step of administering to a person or animal in need of treatment an effective amount of an irradiated cross-linked collagenous biomaterial prepared by a method as claimed in claim 26.

28. A method as claimed in claim 27, wherein the step further includes administering to the person or animal in addition to the irradiated cross-linked collagenous biomaterial at least one component selected from the group consisting of processed bone, insoluble collagenous bone matrix, bone growth inducing factors, cortical bone particles, cancellous bone particles, hydroxyapatites, ceramic powders, demineralized bone particles and mixtures of any two or more thereof.

29. A method as claimed in claim 27, wherein the step of administering comprises administering the cross-linked collagenous material to the site with a syringe.

30. A method as claimed in claim 27, wherein the step of administering comprises employing a haemostatic sponge.

31. A method as claimed in claim 27 wherein the step of administering comprises employing a skin-covering sheet.

32. A tissue regenerating composition including an irradiated cross-linked collagenous biomaterial as claimed in claim 6 and demineralized bone particles.

33. The composition of claim 6, wherein the cross-linked undemineralized collagenous biomaterial has been produced by extracting bone powder.

34. The composition of claim 30, wherein the at least one component comprises demineralized bone particles.

35. A method for preparing a cross-linked collagenous material for a treatment site, comprising irradiating the cross-linked collagenous biomaterial with gamma radiation at a dose of between about 20 and 160 kGy and adding at least one irradiated component selected from the group consisting of processed bone, insoluble collagenous bone matrix, bone growth-inducing factors, cortical bone particles, cancellous bone particles, hydroxyl-apatites, ceramic powders, demineralized bone particles and mixtures of any two or more thereof; and
administering the cross-linked collagenous material to the treatment site.
